(19) United States
(12) Patent Application Publication
(10) Pub. No.: US 2012/0165532 A1
(43) Pub. Date: Jun. 28, 2012

(54) One-Step Synthesis Method of 2,9-Dimethyl-4,7-Diphenyl-1,10- Phenanthroline

(76) Inventors: Lifei Cai, Beijing (CN); Weilong Zhang, Beijing (CN); Hongyu Zhao, Beijing (CN); Lei Dai, Beijing (CN)

(21) Appl. No.: 13/318,716

(22) PCT Filed: Mar. 30, 2010
(86) PCT No.: PCT/CN10/71411

§ 371 Date: Jan. 18, 2012

(30) Foreign Application Priority Data
May 6, 2009 (CN) 200910083628.1

(51) Int. Cl. C07D 471/04

(52) U.S. Cl. 546/88

(57) ABSTRACT

This invention, which belongs to the field of organic synthesis, involves “One-step synthetic method of 2,9-dimethyl-1,7-diphenyl-1,10-phenanthroline”. This method uses O-phenylenediamine and formula III to react under the condition of mixed-shrinking agent, the synthesis can be completed in one step, the stated mixed-shrinking agent is mixture of hydrochloric acid and organic acid. The organic acid serves as phase transfer catalyst and shrinking agent, meanwhile, as buffer reagent, organic acid reduces polymerization of III, and side products as well. The products gained are of high purity and the reaction is mild and easy to control. Since there is no polluting material added and generated, the waste is safe to discharge. In the after treatment of reaction, ketone solvent is used to reduce separation step and product lost, thus improving yield.

### TECHNICAL FIELD

This invention belongs to the field of organic synthesis, and particularly involves efficient synthetic method of 2,9-dimethyl-4,7-diphenyl-1,10-phenanthroline

### TECHNICAL BACKGROUND

1,10-phenanthroline compound is one of the most widely studied and applied nitrogen heterocyclic ring chelating agents. As an important ligand, many complex compounds of it play a significant role in various fields (P. G. Sammes, G. Yahioglu, Chem. Sov. Rev., 1994, 23, 327). Complex compounds with ligands of 1,10-phenanthroline derivatives have good optical properties, and can be used as photosensitizers and photocatalyst (R. Sahai, L. Morgan, D. P. Killema, Inorg. Chem., 1988, 27, 3495). Particularly symmetrically distributed 1,10-phenanthroline derivatives, which are a kind of very important compound worth of study, because these substances can main the two part of ligand symmetrical, and very well avoid stereoisomerism resulted from complexing with metal.

2,9-dimethyl-4,7-diphenyl-1,10-phenanthroline is an important compound belongs to 1,10-phenanthroline derivatives; it can be used to detect copper ion, as well as in the field of photoelectric material. Since 2,9-dimethyl-4,7-diphenyl-1,10-phenanthroline has more than one benzene ring structure, high melting point of 288° C., and energy level 3.3 eV, it is always used as exciton/hole blocking material in OLED. But synthesis of 2,9-dimethyl-4,7-diphenyl-1,10-phenanthroline is not very desirable. Currently, synthetic method of this material is mainly Skraup-Doebner-von-Miller synthesis. This method needs arsenic acid or arsenic pentoxide (Case F. H., Brennan J. A., J. Org. Chem., 1954, 19, 919.). It consists of three violent steps of reaction with only less than 10 percent of total yield; therefore, it is not suitable for industrialization. Defects of these methods are many intermediate products, low yield, and it takes too long to compound a small amount of products, the procedures are complicate, and its performance is poor in terms of economic efficiency. Czech patent CS146030 put forward the one-step synthetic method of 2,9-dimethyl-1,7-diphenyl-1,10-phenanthroline, though it reduced the procedure from three to one, which saved a great deal of manpower, it still used arsenic, which is heavily polluted to the environment, as oxidizing agent; in addition, its repeatability was poor and after treatment was perplexed, thereby, new synthetic method that use no arsenic compound as oxidizing agent is in need. Czech patent CS226921 mentioned using tetrachlorohydroquinone, DDQ and so on as oxidizing agent to synthesize 10-phenanthroline derivatives, though it shook off usage of arsenic compound, great problems were encountered during the process of industrialization of it, such as tetrachlorohydroquinone cannot dissolve in acid solutions and organic solvent, which was hard to treat; in addition, a large amount of products were lost during after treatment, and the yield was only less than 10%. In light of these current synthetic methods, new friendly method with a high yield is in demand.

Introduction of this Invention

Skraup-Doebner-von-Miller synthetic method of 2,9-dimethyl-1,7-diphenyl-1,10-phenanthroline has the problem of low yield and high pollution; while this invention increases yield and production purity by starting from reaction mechanism of synthesis of this kind of compound, choosing pollution-free reactants, and meanwhile, improving reaction conditions to make it more easy to control and ensuring easy after treatment.

The one-step synthetic method of 2,9-dimethyl-1,7-diphenyl-1,10-phenanthroline (see formula I) uses O-phenylenediamine and formula III to react under the condition of mixed-shrinking agent, the synthesis can be completed in one step, the stated mixed-shrinking agent is mixture of hydrochloric acid and organic acid.

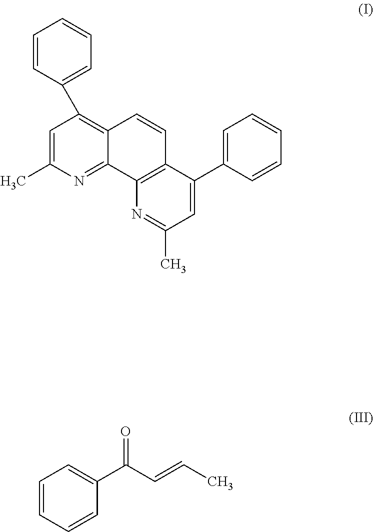

The stated organic acid is one or several of formic acid, acetic acid, propionic acid, butyric acid, valeric acid.

Volumetric proportion of the stated mixture of hydrochloric acid and organic acid is 1:9-9:1.

Volumetric proportion of the stated mixture of hydrochloric acid and organic acid is 3:7-7:3.

The stated synthesis process includes the following procedures: 1) batch addition of formula III into O-phenylenediamine in concentrated hydrochloric acid solution, and react 2-10 hours at 50-90° C.; 2) add organic acid, reflux for 2-10 hours at 90-110° C.

Reaction condition for stated procedure (1) is to react 2-8 hours at 70-85° C., reaction condition for stated procedure (2) is to reflux for 2-8 hours at 90-110° C.

Method for stated procedure (1): add hydrochloric acid in reaction vessel and batch addition of O-phenylenediamine in reaction flask at room temperature, stir 1-6 hours and then add formula III to react.

The stated synthetic reaction also includes procedures of after treatment, in which ammonia water is added in the final reaction solution at 0-5° C., PH is adjusted to 10-13 after that, the water layer is removed, ketones solvent are added, and the solids are separated out, then washed and dried.

The stated ketones solvent is one or several of acetone, methyl acetone, methyl ethyl ketone, 2,5-hexanedione. Based on existing technology, the process of one-step, synthetic method of 2,9-dimethyl-1,7-diphenyl-1,10-phenanthroline is as follows:

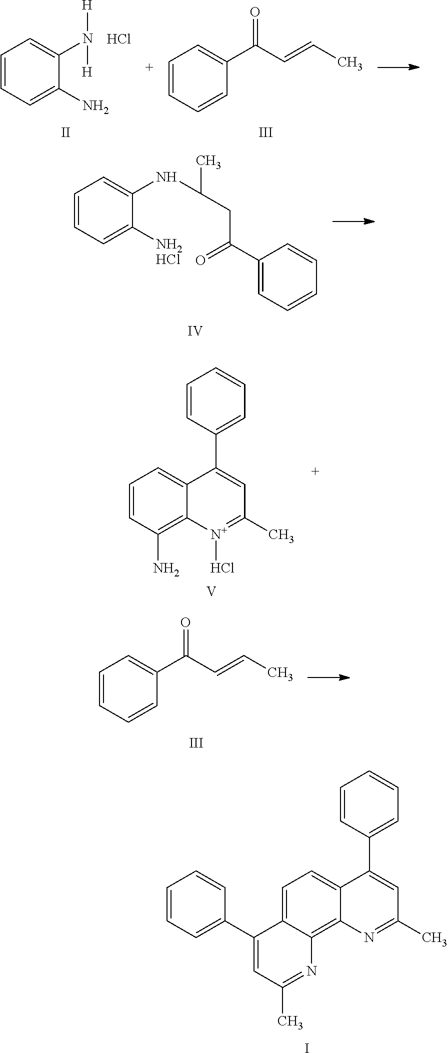

O-phenylenediamine (formula II above is hydrochloride structure), which need to be transformed into hydrochloride in acid environment, is used as starting material in this reaction, because it is easy to react; in sour environment, addition reaction will happen between amino group from formula II and III, generating IV which is quickly converted into V, there is also an amino in compound V which continue to repeat the above reaction with III, then compound I is obtained. As a matter of fact, its reaction mechanism is that O-phenylenediamine reacts with ketene structure and synthesizes I, 10-phenanthroline structure. The technique adopted before used concentrated sulfuric acid, concentrated phosphoric acid as shrinking agent, arsenic pentoxide or arsenic acid as oxidizing agent, and the cyclizaiton and dehydrogenation conditions were severe, therefore, this method had low yield and many side reactions. In order to control yield, two steps of successive reaction have to proceed smoothly, it is also need to choose appropriate oxidizing agent and shrinking agent, and lower reaction conditions. Since compound III has double bond which is prone to gather in strong acid environment, so drop it in the reaction system to reduce generation of side product. After dropping compound III, maintaining temperature unchanged, compound IV, V will be obtained; addition of organic acid promotes condensation reaction and also plays the role of phase transfer. Mixed shrinking agent invented in this innovation reduces reflux temperature and side reaction as well. At the end of this reaction, the product obtained is in the form of hydrochloride which needs to be released with alkali, and ammonia is very economical and effective in this respect. Ketones are used to treat the mixture obtained by dissolving side products in ketones solvent and separating out high purity products. Ketone solvent mentioned is one or several of acetone, methyl acetone, methyl ethyl ketone, 2,5-hexanedione.

During the whole reaction process, the time to add II, III seriously affects product yield, time interval can be 1-6 hours, and suitable time should be 2-4 hours.

Temperature when adding III should be controlled at 50-90° C., favorable temperature is 60-90° C., and the optimal one is 70-85° C.

The patented synthetic process can be carried out in accordance with the following procedures:

(1) Add hydrochloric acid in reaction vessel, and then add II in reaction flask in batch under room temperature, stir 1-6 hours and II will transform to the form of hydrochloride.
(2) Add III at 70-85° C., and react 2-8 hours under this temperature, then the hydrochloride of II will be converted into IV, V, after that, add organic acid in batch.
(3) Raise temperature to 90-100° C. and reflux, after 2-8 hours, the reaction is completed.
(4) Add ammonia in reaction solution at 0-5° C., adjust PH to 8-10, remove water layer, then add ketones solvent, stir and separate out solids, after that, pulling out filters, wash the solids and dry them.

The process above solves the current problem of low yield and many side products and reduces environmental pollution as well, apart from that, it has the following advantages:

(1) O-phenylenediamine dihydrochloride reacts with III under the condition of hydrochloric acid and converts to IV, V, the organic acid serves as phase transfer catalyst and shrinking agent. As buffer reagent, organic acid reduces polymerization of III, and side products as well. The products gained are of high purity and the reaction is mild and easy to control.
(2) Since there is no polluting material added and generated, the waste is safe to discharge.
(3) Ketone solvents can reduce separation step and product lost, thus improving yield.

### DESCRIPTION OF FIGURES

FIG. 1 DSC Spectra of Compounds in Implementation Example 1, melting pointing point of product: 288.77° C.

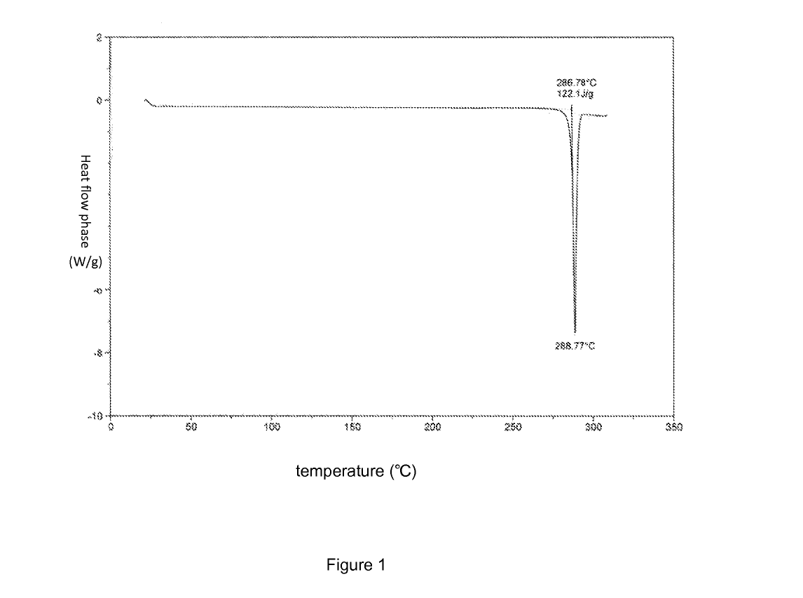

Experiment instrument: 2910MDSCV4.4E, test conditions: 10° C./min, N₂

FIG. 2 DSC Spectra of Compounds in Comparison Example 1, melting pointing

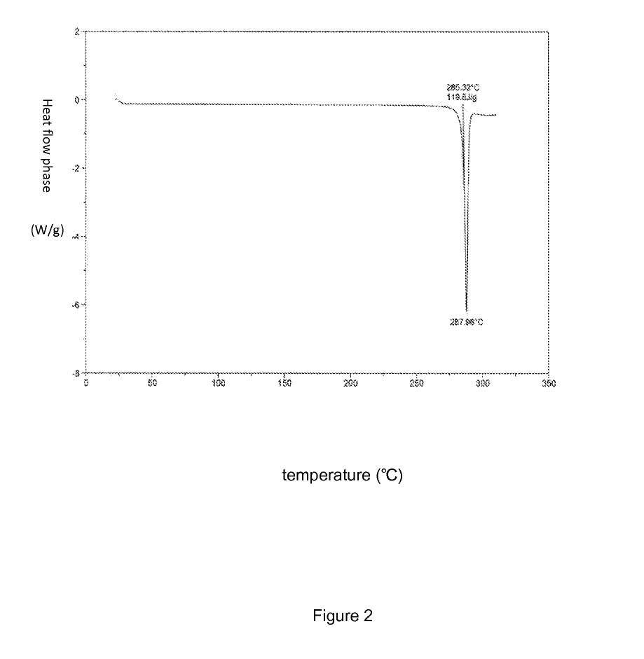

point of product: 287.96° C.

Experiment instrument: 2910MDSCV4.4E, test conditions: 10° C./min, N₂

FIG. 3 TGA Spectra of Compounds in Implementation Example 1

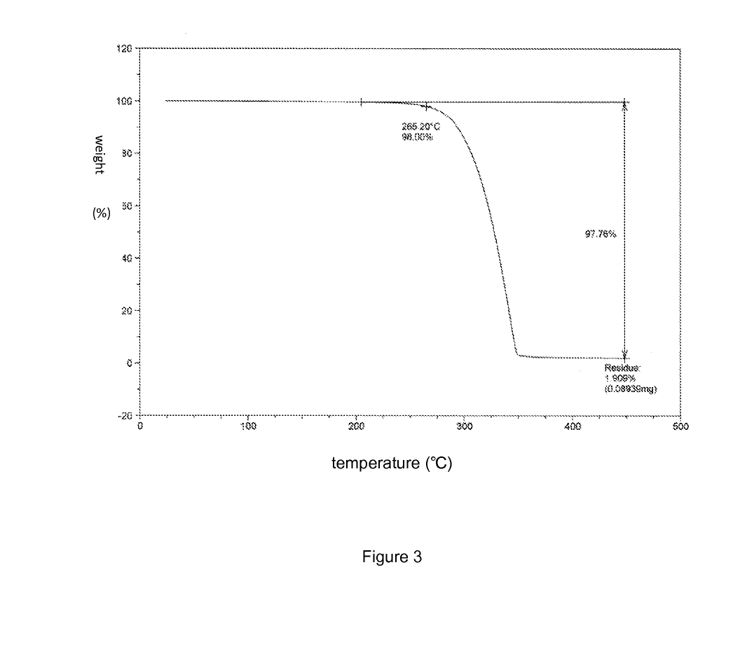

Experiment instrument: TGA Q5000 V3.5 Build 252, test conditions: 10° C./min, N₂

FIG. 4 TGA Spectra of Compounds in Comparison Example 1

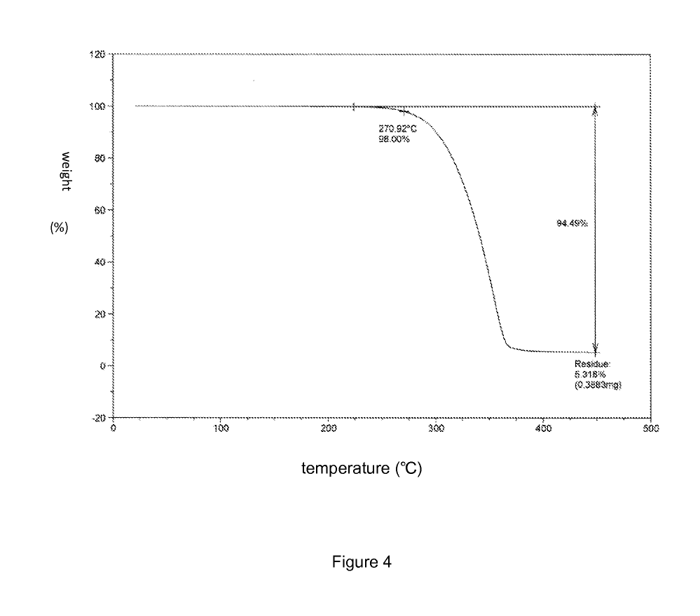

Experiment instrument: TGA Q5000 V3.5 Build 252, test conditions: 10° C./min, N₂

### PRACTICAL IMPLEMENTATION MEASURES

This invention is elaborated below with the help of Implementation Example.

### Preparation of Raw Materials

### 1-1: (Preparation of Crotonic Acid Chloride)

First add 1 kg crotonic acid to a 5 L reaction flask, and then add 1650 mL (1.5 mol) thionyl chloride while stirring slowly, a large amount of HCl will be produced. Stir at room temperature until there is no violent emission of gas, and then raise temperature slowly until reflux. Atmospheric distillation is carried out after the reaction is finished, then collect 871 g fraction at 116-124° C., the yield is 72%.

### 1-2: (Preparation of Compound III)

Add 46 L benzene in a 100 L reaction flask, and then add 21.75 kg anhydrous aluminum chloride in it in batch while stirring. Cool it to 0-5° C., drop 11.6 kg crotonic acid chloride and keep the temperature below 5° C., a large amount of bright yellow solids will appear in the flask, and the amount will increase gradually. After all crotonic acid chloride is added, maintain the temperature at 5-10° C. and stir until no HCl gas is generated, then pour the reactant into mixture of ice and concentrated hydrochloric acid (3:1) in batch, the bright yellow solids will dissolve. Wait until the reactant is stationary, it can be seen that it separates into two layers, the pale yellow upper layer is organic layer, the slightly turbid lower layer is water layer. Extract the water layer 2 times with 20 L ethyl acetate, wash benzene layer and ethyl acetate twice with SOL saturated salt water, and wash the organic layer to neutral with 10% sodium hydroxide solution, then again wash twice with saturated salt water, fully dry it with anhydrous sodium sulfate. Reduced pressure distillation is carried out to remove low boiling point solvent, then repeat reduced pressure distillation and collect 10.5 kg fractions at 144-164° C., the yield is 66%, pale yellow oily liquid is obtained with yield of 80%.

### Implementation Example 1

### Method of this Invention

First add 6.51 g concentrated hydrochloric acid, stir and expose it to nitrogen for half an hour. Add 470 g O-phenylenediamine in batch, stir 2 hours at 60° C., then add 1.3 kg poly(vinyl phenyl ketone) at 70-85° C. and stir 3 hours, while stirring, add 5 L acetic acid in batch. Raise temperature to about 94° C. slowly to reflux for 8 hours, the reaction solution is brown. Stop heating, wait until it reaches room temperature, and then cool it by ice bath, add 6 kg ice cube to the reaction solution in batch while stirring, then add ammonia slowly, adjust PH to 8-10, and remove the upper water layer.

Add 6 L acetone in the black viscous substance and stir 1 hour, and then 300 g yellow filter cake is gained after filtration. After recrystallization, 230 g product with 99% purity is obtained, the yield is 14%.

HNMR (CDCl₃, 400 MHz) δ (ppm):3.1 (s, 3H), 7.56 (s, 1H), 7.62 (m, 5H), 7.86 (s, 1H).

MS (EI) 360

### Implementation Example 2

### Amplified in Accordance with Patent

Amplify the experiment 10 times in accordance with the method of Implementation Example 1, the yield is 14-20%, the reaction waste liquid can be discharged since it contains no harmful substance.

### Comparison Example 1

### Arsenic Pentoxide Method, Provided by Literature

First add 10 L concentrated hydrochloric acid, stir and expose it to nitrogen for half an hour. Then add 855 g O-phenylenediamine in batch, stir 2 hours at 60° C., add 2630 g poly(vinyl phenyl ketone) at 70-85° C. and stir 3 hours, while stirring, add 1883 g arsenic pentoxide in batch. Raise temperature to about 110° C. slowly to reflux for 8 hours, the reaction solution is brown. Stop heating, wait until it reaches room temperature, and then cool it by ice bath, add 10 kg ice cube to the reaction solution in batch while stirring, then add ammonia slowly, adjust PH to 8-10, and remove the upper water layer.

Add 10 L acetone in the black viscous substance and stir 1 hour, and then 300 g yellow filter cake is gained after filtration. Put the filter cake in a vessel, add 4 L methylene chloride and stir 1 hour, after that, filtrate it and process methylene chloride filtrate through rotary evaporation, add acetone after dried, filtrate it again and 287.68 g product of 96% purity is obtained, purify over 98% product, the yield is 8%.

HNMR (CDCl₃, 400 MHz) δ (ppm):3.1 (s, 3H), 7.56 (s, 1H), 7.62 (m, 5H), 7.86 (s, 1H).

MS (EI) 360

### Comparison Example 2

### Tetrachloro-Hydroquinone Method

First add 500 ml concentrated hydrochloric acid, stir and expose it to nitrogen for half an hour. Then add 27 g O-phenylenediamine in batch, stir 2 hours at 60° C., add 80 g poly(vinyl phenyl ketone) at 70-85° C. and stir 3 hours, while stirring, add 135 g tetrachloro-hydroquinone in batch. Raise temperature to about 94° C. slowly to reflux for 8 hours, the reaction solution is brown. Stop heating, wait until it reaches room temperature, and then cool it by ice bath, add 500 g ice cube to the reaction solution in batch while stirring, then add ammonia slowly, adjust PH to 8-10, and remove the upper water layer.

Add 600 ml acetone in the black viscous substance and stir 1 hour, and then yellow filter cake is gained after filtration. After recrystallization, 13 g product with 99% purity is obtained, the yield is 12%.

HNMR (CDCl₃, 400 MHz) δ (ppm):3.1 (s, 3H), 7.56 (s, 1H), 7.62 (m, 5H), 7.86 (s, 1H).

MS (EI) 360

### Comparison Example 3

### Amplified Tetrachloro-Hydroquinone Method

Amplify the experiment 10 times in accordance with the method of Comparison Example 2, after cooling, agglomeration appears which cannot be dealt with, the solid cannot dissolve even if reheated; a large amount of insoluble solid is generated because side product of tetrachloro-hydroquinone contains abundant organic chloride which is heavily polluted to the environment.

1. A one-step synthetic method of 2,9-dimethyl-1,7-diphenyl-1,10-phenanthroline uses o-phenylenediamine and formula III to react under the condition of mixed-shrinking agent, the synthesis can be completed in one step, the stated mixed-shrinking agent is mixture of concentrated hydrochloric acid and organic acid.

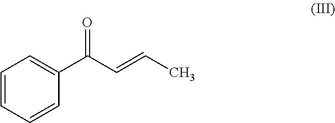

2. The one-step synthetic method according to claim 1, the stated organic acid is one or several of formic acid, acetic acid, propionic acid, butyric acid, valeric acid.

3. The one-step synthetic method according to claim 2, volumetric proportion of the stated mixture of hydrochloric acid and organic acid is 1:9 to 9:1.

4. The one-step synthetic method according to claim 3, volumetric proportion of the stated mixture of hydrochloric acid and organic acid is 3:7 to 7:3.

5. The one-step synthetic method according to claim 1, the stated synthesis process includes the following procedures: 1) add formula III to O-phenylenediamine in concentrated hydrochloric acid solution, and react 2-10 hours at 50-90° C.; 2) add organic acid, reflux for 2-10 hours at 90-110° C.

6. The one-step synthetic method according to claim 5, reaction condition for procedure (1) is to react 2-8 hours at 70-85° C.

7. The one-step synthetic method according to claim 5, reaction condition for procedure (2) is to reflux for 2-8 hours at 90-110° C.

8. The one-step synthetic method according to claim 5, the concentrated hydrochloric acid solution of o-phenylenediamine in stated procedure (1) is obtained as follows:
add hydrochloric acid in reaction vessel, and then add o-phenylenediamine in reaction flask in batch under room temperature, stir 1-6 hours.

9. The one-step synthetic method according to claim 1, the stated synthetic reaction also includes after treatment, which is to add ammonia to the final reaction solution at 0-5° C., adjust PH to 10-13, remove water layer, and then add ketones solvent and separate out solids, after that, leaching, wash and dry them.

10. The one-step synthetic method according to claim 9, the stated ketones solvent is one or several of acetone, methyl acetone, methyl ethyl ketone, 2,5-hexanedione.
